(19) United States
(12) Patent Application Publication
(10) Pub. No.: US 2021/0113579 A1
(43) Pub. Date: Apr. 22, 2021

(54) FLUOROQUINOLONE FORMULATIONS FOR CYSTIC FIBROSIS

(71) Applicant: Horizon Orphan LLC, Lake Forest, IL (US)

(72) Inventor: Donald VANDERVANTER, Puyallup, WA (US)

(21) Appl. No.: 16/971,945

(22) PCT Filed: Feb. 2, 2017
(86) PCT No.: PCT/US2017/016161

§ 371 Date: Aug. 21, 2020

Related U.S. Application Data
(60) Provisional application No. 62/348,439, filed on Jun. 10, 2016.
(60) Provisional application No. 62/293,554, filed on Feb. 10, 2016.
(60) Provisional application No. 62/292,065, filed on Feb. 5, 2016.

(51) Int. Cl. A61K 31/5383; A61K 9/00; A61K 47/02; A61K 31/496; A61K 31/7036; A61K 31/427; A61P 31/04; A61K 31/4375; A61K 31/4709; A61P 11/00

(52) U.S. Cl. CPC A61K 31/5383; A61K 9/0073; A61K 47/02; A61K 31/496; A61P 11/00; A61K 31/427; A61P 31/04; A61K 31/4375; A61K 31/4709; A61K 31/7036

(57) ABSTRACT

The disclosure provides methods for treating cystic fibrosis with fluoroquinolone formulations.

### CROSS-REFERENCE TO RELATED APPLICATIONS

This application claims priority to U.S. Application No. 62/348,439 filed Jun. 10, 2016; U.S. Application No. 62/293,554 filed Feb. 10, 2016; and U.S. Application No. 62/292,065 filed Feb. 5, 2016; the disclosures of each of which are incorporated by reference herein in their entirety.

### BACKGROUND

Cystic fibrosis lung disease is characterized by chronic respiratory tract infection with multiple bacterial species frequently dominated by Pseudomonas aeruginosa, which has been associated with accelerated lung disease progression, increased morbidity, and decreased survival. Chronic Pseudomonas aeruginosa infection is typically treated with chronic inhaled antibiotics to suppress infection, reduce risk of pulmonary exacerbations, improve quality of life, and preserve lung function.

Despite the availability of several inhaled antimicrobial classes, there is need for additional safe and effective alternative options. The response to aerosolized tobramycin, as assessed by change in spirometry, becomes attenuated after extended exposure, a phenomenon that is not explained by selection of bacterial populations with decreased in vitro tobramycin susceptibilities. It is likely that a similar attenuation of efficacy will occur for other inhaled antimicrobials with extended exposure. In addition some patients are unable to tolerate particular inhaled antibiotic formulations while others may find an inhaled therapy an excessive treatment burden resulting in poor adherence. Thus, there is a need for additional inhaled antibiotic options; including additional antimicrobial classes to allow for greater rotation of therapies and potential for extension of the effective lives of all inhaled antibiotic classes.

Antibiotic classes currently approved in many countries for use by inhalation include the aminoglycosides (tobramycin), monobactams (aztreonam) and polymyxins (colistimethate). A separate antibiotic class with high potency and a broad spectrum of action, fluoroquinolones, is used extensively as oral and intravenous formulations to treat cystic fibrosis lung disease.

There is a need in the art for new and improved antibiotic therapies to treat cystic fibrosis. The disclosure is directed to this, as well as other, important ends.

### SUMMARY

Described herein are methods to treat cystic fibrosis in high-risk patients by administering a therapeutically effective amount of an aerosol of a solution comprising a fluoroquinolone, such as levofloxacin or ofloxacin.

Described herein are methods to treat pulmonary bacterial infections in high-risk cystic fibrosis patients by administering a therapeutically effective amount of an aerosol of a solution comprising a fluoroquinolone, such as levofloxacin or ofloxacin.

Described herein are methods to treat Pseudomonas aeruginosa pulmonary infections in high-risk cystic fibrosis patients by administering a therapeutically effective amount of an aerosol of a solution comprising a fluoroquinolone, such as levofloxacin or ofloxacin.

Described herein are methods to treat pulmonary exacerbations in high-risk cystic fibrosis patients by administering a therapeutically effective amount of an aerosol of a solution comprising a fluoroquinolone, such as levofloxacin or ofloxacin.

Described herein are methods to reduce the incidents of pulmonary exacerbations in high-risk cystic fibrosis patients by administering a therapeutically effective amount of an aerosol of a solution comprising a fluoroquinolone, such as levofloxacin or ofloxacin.

Described herein are methods to extend the time between the onset of pulmonary exacerbations in high-risk cystic fibrosis patients by administering a therapeutically effective amount of an aerosol of a solution comprising a fluoroquinolone, such as levofloxacin or ofloxacin.

Described herein are methods to reduce a Pseudomonas aeruginosa sputum density in high-risk cystic fibrosis patients by administering a therapeutically effective amount of an aerosol of a solution comprising a fluoroquinolone, such as levofloxacin or ofloxacin.

These and other aspects are described in more detail below.

### BRIEF DESCRIPTION OF THE FIGURES

FIG. 1 shows the proportion of patients who did not experience a pulmonary exacerbation over the course of the 28 day treatment, and beyond to 70 days, in the levofloxacin inhalation solution (LIS) treatment group and placebo group.

FIG. 2A shows the LS mean absolute change in FEV₁ over the course of the 28 day treatment (shaded area), and over the course of days 29-56 after treatment (non-shaded area), in the levofloxacin inhalation solution (LIS) treatment group and placebo group. Differences between treatment groups were statistically significant at Day 28. FIG. 2B shows the LS mean change sputum Pseudomonas aeruginosa over the course of the 28 day treatment (shaded area), and over the course of days 29-56 after treatment (non-shaded area) in the levofloxacin inhalation solution (LIS) treatment group and placebo group.

### DETAILED DESCRIPTION

As used in this specification and the appended claims, the singular forms “a,” “an” and “the” include plural referents unless the context clearly dictates otherwise. Thus, for example, “an active agent” refers not only to a single active agent but also to a combination of two or more different active agents, “a dosage form” refers to a combination of dosage forms as well as to a single dosage form, and the like.

Unless defined otherwise, all technical and scientific terms used herein have the meaning commonly understood by one of ordinary skill in the art to which the invention pertains. Specific terminology of particular importance to the description of the present invention is defined below

“Patient” refers to mammals, preferably humans. In embodiments, the human is an adult. In embodiments, the human is an adolescent. In embodiments, the human is a child. In embodiments, the human is an infant. In embodiments, the patient has cystic fibrosis, preferably a human with cystic fibrosis. “Patient” also refers to a human with cystic fibrosis on an antibiotic treatment (other than levofloxacin or ofloxacin) who has symptoms or pulmonary bacterial infections that are not adequately controlled on the antibiotic treatment (other than levofloxacin or ofloxacin), such that the patent requires a new antibiotic therapy such as that described herein.

“High-risk patient” or “high-risk cystic fibrosis patient” refers to a patient that experienced at least 2 occurrences of a pulmonary exacerbation in the prior year. In embodiments, a “high-risk patient” or “high-risk cystic fibrosis patient” refers to a patient that experienced at least 3 occurrences of a pulmonary exacerbation in the prior year. In embodiments, a “high-risk patient” or “high-risk cystic fibrosis patient” refers to a patient that experienced at least 4 occurrences of a pulmonary exacerbation in the prior year. In embodiments, a “high-risk patient” or “high-risk cystic fibrosis patient” refers to a patient that experienced at least 5 occurrences of a pulmonary exacerbation in the prior year. In embodiments, a “high-risk patient” or “high-risk cystic fibrosis patient” refers to a patient that experienced at least 6 occurrences of a pulmonary exacerbation in the prior year. In embodiments, a “high-risk patient” or “high-risk cystic fibrosis patient” refers to a patient that experienced at least 7 occurrences of a pulmonary exacerbation in the prior year.

“Pulmonary exacerbation” is a pulmonary illness which results in a clinician's decision to treat the cystic fibrosis patient with an antibiotic for that pulmonary illness. The antibiotic can be an oral antibiotic, an intravenous antibiotic, or an inhaled antibiotic. In embodiments, the antibiotic is an intravenous antibiotic or an inhaled antibiotic. In embodiments, the antibiotic is an intravenous antibiotic. In embodiments, the antibiotic is an inhaled antibiotic.

The “pulmonary illness” which results in a clinician's decision to treat the patient with an antibiotic can be any pulmonary illness caused by or related to cystic fibrosis. In embodiments, the pulmonary illness which results in a clinician's decision to treat the patient with an antibiotic occurs when the patient is diagnosed as having at least four of the twelve Fuchs criteria. In embodiments, the pulmonary illness which results in a clinician's decision to treat the patient with an antibiotic is a a pulmonary bacterial infection. In embodiments, the pulmonary illness which results in a clinician's decision to treat the patient with an antibiotic is a Pseudomonas pulmonary bacterial infection. In embodiments, the pulmonary illness which results in a clinician's decision to treat the patient with an antibiotic is a Pseudomonas aeruginosa pulmonary bacterial infection. In embodiments, the pulmonary illness which results in a clinician's decision to treat the patient with an antibiotic is a Staphylococcus pulmonary bacterial infection. In embodiments, the pulmonary illness which results in a clinician's decision to treat the patient with an antibiotic is a Staphylococcus aureus pulmonary bacterial infection. In embodiments, the pulmonary illness which results in a clinician's decision to treat the patient with an antibiotic is a Burkholderia pulmonary bacterial infection. In embodiments, the pulmonary illness which results in a clinician's decision to treat the patient with an antibiotic is a Burkholderia cepacia pulmonary bacterial infection. In embodiments, the pulmonary illness which results in a clinician's decision to treat the patient with an antibiotic is a Pseudomonas pulmonary bacterial infection, Staphylococcus pulmonary bacterial infection, a Burkholderia pulmonary bacterial infection, or a combination of two or more thereof. In embodiments, the pulmonary illness which results in a clinician's decision to treat the patient with an antibiotic is a Pseudomonas aeruginosa pulmonary bacterial infection, a Staphylococcus aureus pulmonary bacterial infection, a Burkholderia cepacia pulmonary bacterial infection, or a combination of two or more thereof. In embodiments, the pulmonary illness which results in a clinician's decision to treat the patient with an antibiotic is a 10% or more decrease in a patient's forced expiratory volume in 1 second (FEV₁) of the predicted value using Hankinson/NHANES III reference equations. In embodiments, the pulmonary illness which results in a clinician's decision to treat the patient with an antibiotic is any pulmonary disease, dysfunction, or disorder that can occur in a patient with cystic fibrosis.

“Fuchs criteria” refers to the symptoms described in Fuchs et al, The New England Journal of Medicine, 331:637-642 (1994). The twelve symptoms that fall within the scope of the Fuchs criteria include: (i) change in sputum; (ii) new or increased hemoptysis; (iii) increased cough; (iv) increased dyspnea; (v) malaise, fatigue, or lethargy; (vi) temperature above 38° C.; (vii) anorexia or weight loss; (viii) sinus pain or tenderness; (ix) change in sinus discharge; (x) change in physical examination of the chest; (xi) decrease in pulmonary function by 10% or more from a previously recorded value; and (xii) radiographic change indicative of pulmonary infection. In embodiments, the change in sputum is an increase in sputum. In embodiments, the change in sputum is a decrease in sputum. In embodiments, the change in physical examination of the chest is a negative change, a deteriorating change, or a worsening change.

“Pulmonary bacterial infection” refers to any pulmonary bacterial infection that can occur in a patient with cystic fibrosis. In embodiments, the pulmonary bacterial infection is a Pseudomonas pulmonary bacterial infection. In embodiments, the pulmonary bacterial infection is a Pseudomonas aeruginosa pulmonary bacterial infection. In embodiments, pulmonary bacterial infection is a Staphylococcus pulmonary bacterial infection. In embodiments, the pulmonary bacterial infection is a Staphylococcus aureus pulmonary bacterial infection. In embodiments, the pulmonary bacterial infection is a Burkholderia pulmonary bacterial infection. In embodiments, the pulmonary bacterial infection is a Burkholderia cepacia pulmonary bacterial infection. In embodiments, the pulmonary bacterial infection is a Pseudomonas pulmonary bacterial infection, a Staphylococcus pulmonary bacterial infection, a Burkholderia pulmonary bacterial infection, or a combination of two or more thereof. In embodiments, the pulmonary bacterial infection is a Pseudomonas aeruginosa pulmonary bacterial infection, a Staphylococcus aureus pulmonary bacterial infection, a Burkholderia cepacia pulmonary bacterial infection, or a combination of two or more thereof.

“Clinician” refers to any person who can prescribe medications (e.g., antibiotics) or treat cystic fibrosis patients, such as doctors, physician assistants, nurses, clinical investigators, pharmacists, and the like.

“Prior year” refers to the 12 months prior to initiation of treatment with a levofloxacin inhalation solution, ofloxacin inhalation solution, or fluoroquinolone inhalation solution as described herein.

“Levofloxacin inhalation solution” refers to an aerosol of a solution of levofloxacin. In embodiments, levofloxacin inhalation solution is an aerosol of a solution of levofloxacin and a divalent or trivalent cation. In embodiments, levofloxacin inhalation solution is an aerosol of a solution of levofloxacin and a divalent cation. In embodiments, levofloxacin inhalation solution is an aerosol of a solution of about 75 mg/ml to about 150 mg/ml of levofloxacin and about 150 mM to about 250 mM of a divalent cation. In embodiments, levofloxacin inhalation solution is an aerosol of a solution of about 80 mg/ml to about 120 mg/ml of levofloxacin and about 180 mM to about 220 mM of a divalent cation. In embodiments, levofloxacin inhalation solution is an aerosol of a solution of about 90 mg/ml to about 110 mg/ml of levofloxacin and about 175 mM to about 225 mM of a magnesium cation. In embodiments, levofloxacin inhalation solution is an aerosol of a solution of about 100 mg/ml of levofloxacin and about 40 mg/ml of magnesium chloride. In embodiments, levofloxacin inhalation solution is an aerosol of an aqueous solution of about 102.50 mg/ml of levofloxacin hemihydrate and about 40.66 mg/ml magnesium chloride hexahydrate, e.g., QUINSAIR®, Raptor Pharmaceuticals, Inc. In embodiments, the levofloxacin inhalation solution may optionally contain pharmaceutically acceptable excipients. In embodiments, the levofloxacin inhalation solution is administered with a nebulizer (e.g., PARI eFlow® Rapid Nebulizer System). Exemplary levofloxacin inhalation solutions are described in U.S. Pat. Nos. 7,838,532, 8,629,139, and 8,815,838, the disclosures of which are incorporated by reference herein in their entirety. In embodiments, the levofloxacin inhalation solution is administered to patients whose symptoms or pulmonary bacterial infections are not adequately controlled by another antibiotic therapy. This administration can be in place of the other antibiotic therapy or in addition to the other antibiotic therapy.

“Ofloxacin inhalation solution” refers to an aerosol of a solution of ofloxacin. In embodiments, ofloxacin inhalation solution is an aerosol of a solution of ofloxacin and a divalent or trivalent cation. In embodiments, ofloxacin inhalation solution is an aerosol of a solution of about 75 mg/ml to about 150 mg/ml of ofloxacin and about 150 mM to about 250 mM of a divalent cation. In embodiments, ofloxacin inhalation solution is an aerosol of a solution of about 100 mg/ml of ofloxacin and about 200 mM magnesium chloride. In embodiments, the ofloxacin inhalation solution may optionally contain pharmaceutically acceptable excipients. In embodiments, the ofloxacin inhalation solution is administered with a nebulizer (e.g., PARI eFlow® Rapid Nebulizer System). Exemplary ofloxacin inhalation solutions are described in U.S. Pat. Nos. 7,838,532, 8,629,139, and 8,815,838, the disclosures of which are incorporated by reference herein in their entirety. In embodiments, the ofloxacin inhalation solution is administered to patients whose symptoms or pulmonary bacterial infections are not adequately controlled by another antibiotic therapy. This administration can be in place of the other antibiotic therapy or in addition to the other antibiotic therapy.

“Fluoroquinolone inhalation solution” refers to an aerosol of a solution of a fluoroquinolone. In embodiments, fluoroquinolone inhalation solution is an aerosol of a solution of levofloxacin and a divalent or trivalent cation. In embodiments, fluoroquinolone inhalation solution is an aerosol of a solution of about 50 mg/ml to about 300 mg/ml of a fluoroquinolone and about 50 mM to about 400 mM of a divalent or trivalent cation. In embodiments, the fluoroquinolone inhalation solution may optionally contain pharmaceutically acceptable excipients. In embodiments, the fluoroquinolone inhalation solution is administered with a nebulizer (e.g., PARI eFlow® Rapid Nebulizer System). Exemplary fluoroquinolone inhalation solutions are described in U.S. Pat. No. 7,838,532, the disclosure of which is incorporated by reference herein in its entirety.

“Divalent cation” refers to cations of magnesium, calcium, zinc, copper, and iron. In one embodiment, divalent cation is a cation of magnesium.

“Trivalent cation” refers to cations of aluminum and iron.

“Fluoroquinolone” refers to fluoroquinolone antibiotics which include, e.g., levofloxacin, ofloxacin, ciprofloxacin, enoxacin, gatifloxacin, gemifloxacin, lomefloxacin, moxifloxacin, norfloxacin, pefloxacin, sparfloxacin, garenoxacin, sitagloxacin, and DX-619 (i.e., 7-[(3R)-3-(1-aminocyclopropyl)pyrrolidin-1-yl]-1-[(1R,2S)-2-fluorocyclopropyl]-8-methoxy-4-oxoquinoline-3-carboxylic acid). In embodiments, fluoroquinolone is levofloxacin or ofloxacin. In embodiments, fluoroquinolone is levofloxacin. In embodiments, the fluoroquinolone is ofloxacin. In embodiments, the fluoroquinolone is ciprofloxacin, enoxacin, gatifloxacin, gemifloxacin, lomefloxacin, moxifloxacin, norfloxacin, pefloxacin, sparfloxacin, garenoxacin, sitagloxacin, or DX-619.

“Pharmaceutically acceptable excipient” refers to a substance that aids the administration of an active agent to and absorption by a patient and can be included in the inhalation solutions described herein without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful herein.

The term “pharmaceutically acceptable salts” is meant to include salts of the active compounds, divalent cations, and trivalent cations that are prepared with relatively nontoxic acids or bases, depending on the particular substituents found on the compounds described herein. When compounds contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. In embodiments, the divalent and trivalent cations are in the form of pharmaceutically acceptable salts, e.g., magnesium chloride, magnesium sulfate, and the like.

The terms “treating” or “treatment” refer to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. For example, certain methods herein may treat cystic fibrosis by treating a pulmonary exacerbation and/or treating a pulmonary illness, such as a pulmonary bacterial infection (e.g., Pseudomonas aeruginosa pulmonary bacterial infections), reducing the incidents of pulmonary exacerbations, extending the time between the occurrence of pulmonary exacerbations, and/or reducing the severity of pulmonary exacerbations.

The phrase “reducing the incidents of pulmonary exacerbations” or equivalent phrase means that a patient will have fewer incidents of pulmonary exacerbations in the future compared to a similar patient in the same situation (or the same patient) who does not receive the treatments described herein, compared to the absence of treatment, or compared to some other acceptable control. “Reducing the incidents of pulmonary exacerbations” includes reducing the incidents during the course of the treatments described herein, reducing the incidents after the course of the treatments described herein ends, or reducing the incidents during the course of treatments described herein and after the course of the treatments end.

The phrase “extend the time between the occurrence of pulmonary exacerbations” or equivalent phrase means that a patient will experience a longer period of time between the occurrence of future pulmonary exacerbations compared to a similar patient in the same situation (or the same patient) who does not receive the treatments described herein, compared to the absence of treatment, or compared to some other acceptable control. “Extending the time between the occurrences of pulmonary exacerbations” or equivalent phrase includes extending the time during the course of the treatments described herein, extending the time after the course of the treatments described herein ends, or extending the time during the course of treatments described herein and after the course of the treatments end.

The phrase “reducing the severity of pulmonary exacerbations” or equivalent phrase means that a patient may experience a pulmonary exacerbation, but the duration of the pulmonary exacerbation will be for a shorter period of time (e.g., compared to a similar patient in the same situation (or the same patient) who does not receive the treatments described herein, compared to the absence of treatment, or compared to some other acceptable control) and/or the number of Fuchs criteria that they experience will be lower (e.g., compared to a similar patient in the same situation (or the same patient) who does not receive the treatments described herein, compared to the absence of treatment, or compared to some other acceptable control) and/or the course of treatment for the pulmonary exacerbation will be for a shorter period of time (e.g., compared to a similar patient in the same situation (or the same patient) who does not receive the treatments described herein, compared to the absence of treatment, or compared to some other acceptable control). “Reducing the severity of pulmonary exacerbations” includes reducing the severity during the course of the treatments described herein, reducing the severity after the course of the treatments described herein ends, or reducing the severity during the course of treatments described herein and after the course of the treatments end.

The terms “administration” and “administering” refer to the methods of providing the levofloxacin inhalation solutions, ofloxacin inhalation solutions, or fluoroquinolone inhalation solutions to a patient. In embodiments, the solutions described herein are administered topically. In embodiments, the solutions described herein are administered to the lungs of the patient, e.g., by pulmonary administration through the use of an appropriate device, such as a nebulizer. In embodiments, the levofloxacin inhalation solutions, ofloxacin inhalation solutions, or fluoroquinolone inhalation solutions are administered to a patient whose symptoms or pulmonary bacterial infections are not adequately controlled by a different antibiotic therapy. The administration can be a replacement of the different antibiotic therapy.

“Dosing regimen” refers to the time between administration of two doses of a levofloxacin inhalation solution, ofloxacin inhalation solution, or a fluoroquinolone inhalation solution to a patient. In embodiments, levofloxacin inhalation solution, ofloxacin inhalation solution, or a fluoroquinolone inhalation solution are administered daily for one month (e.g., 28 days) of treatment, then the patient stops treatment for one month (e.g., 28 days), and then resumes treatment for another month (e.g., 28 days). Treatment cycles may be repeated for as long as the patient benefits. In embodiments, the levofloxacin inhalation solution, ofloxacin inhalation solution, or a fluoroquinolone inhalation solution are administered once daily or twice daily.

“Dose” refers to the amount (e.g., mg) or concentration (e.g., mg/ml) of a fluoroquinolone (e.g., levofloxacin or ofloxacin) that is administered to a patient or that is contained in a formulation. In embodiments, the dose of the fluoroquinolone (e.g., levofloxacin or ofloxacin) is from about 25 mg to about 400 mg; about 25 mg to about 300 mg; about 25 mg to about 200 mg; or from about 50 mg to about 150 mg; or from about 75 mg to about 125 mg; or from about 90 mg to about 110 mg. In embodiments, the dose of the fluoroquinolone (e.g., levofloxacin or ofloxacin) is about 100 mg. In embodiments, the dose of the fluoroquinolone (e.g., levofloxacin or ofloxacin) is from about 100 mg to about 300 mg; or from about 200 mg to about 300 mg; or from about 210 mg to about 270 mg. In embodiments, the dose of the fluoroquinolone (e.g., levofloxacin or ofloxacin) is about 220 mg to about 260 mg. In embodiments, the dose of the fluoroquinolone (e.g., levofloxacin or ofloxacin) is about 230 mg to about 250 mg. In embodiments, the dose of the fluoroquinolone (e.g., levofloxacin or ofloxacin) is about 240 mg.

The disclosure provides methods to treat pulmonary bacterial infections in high-risk cystic fibrosis patients by administering a therapeutically effective amount of an aerosol of a solution comprising a fluoroquinolone. In embodiments, the methods to treat the pulmonary bacterial infections further comprise reducing the incidents of future pulmonary bacterial infections in the high-risk cystic fibrosis patients. In embodiments, the methods to treat the pulmonary bacterial infections further comprise increasing or extending the time between the onset of future pulmonary bacterial infections in the high-risk cystic fibrosis patients. In embodiment, the methods to treat the pulmonary bacterial infections further comprise reducing the incidents of future pulmonary bacterial infections and increasing or extending the time between the onset of future pulmonary bacterial infections in the high-risk cystic fibrosis patients. In embodiments, the methods to treat the pulmonary bacterial infections further comprise reducing the severity of the pulmonary bacterial infections in the high-risk cystic fibrosis patients. In embodiments, the methods to treat pulmonary bacterial infections further comprise reducing the incidents of future pulmonary exacerbations in the high-risk cystic fibrosis patients. In embodiments, the methods to treat pulmonary bacterial infections further comprise increasing or extending the time between the onset of future pulmonary exacerbations in the high-risk cystic fibrosis patients. In embodiment, the methods to treat pulmonary bacterial infections further comprise reducing the incidents of future pulmonary exacerbations and increasing or extending the time between the onset of future pulmonary exacerbations in the high-risk cystic fibrosis patients. In embodiments, the methods to treat pulmonary bacterial infections further comprise reducing the severity of pulmonary exacerbations in the high-risk cystic fibrosis patients. In embodiments, the pulmonary bacterial infection is a chronic pulmonary bacterial infection. In embodiments, the fluoroquinolone is levofloxacin. In embodiments, the fluoroquinolone is ofloxacin. In embodiments, the high-risk patient experienced at least 3 occurrences of a pulmonary exacerbation in the prior year. In embodiments, the aerosol of the solution i administered to a patient whose symptoms or pulmonary bacterial infections are not adequately controlled by a different antibiotic therapy.

The disclosure provides methods to treat Pseudomonas aeruginosa pulmonary infections in high-risk cystic fibrosis patients by administering a therapeutically effective amount of an aerosol of a solution comprising a fluoroquinolone. In embodiments, the methods to treat the Pseudomonas aeruginosa pulmonary infection further comprise reducing the incidents of future Pseudomonas aeruginosa pulmonary infections in the high-risk cystic fibrosis patients. In embodiments, the methods to treat the Pseudomonas aeruginosa pulmonary infection further comprise increasing or extending the time between the onset of future Pseudomonas aeruginosa pulmonary infections in the high-risk cystic fibrosis patients. In embodiment, the methods to treat the Pseudomonas aeruginosa pulmonary infection further comprise reducing the incidents of future Pseudomonas aeruginosa pulmonary infections and increasing or extending the time between the onset of future Pseudomonas aeruginosa pulmonary infections in the high-risk cystic fibrosis patients. In embodiments, the methods to treat the Pseudomonas aeruginosa pulmonary infection further comprise reducing the severity of the Pseudomonas aeruginosa pulmonary infections in the high-risk cystic fibrosis patients. In embodiments, the methods to treat the Pseudomonas aeruginosa pulmonary infection further comprise reducing the incidents of future pulmonary exacerbations in the high-risk cystic fibrosis patients. In embodiments, the methods to treat the Pseudomonas aeruginosa pulmonary infection further comprise increasing or extending the time between the onset of future pulmonary exacerbations in the high-risk cystic fibrosis patients. In embodiment, the methods to treat the Pseudomonas aeruginosa pulmonary infection further comprise reducing the incidents of future pulmonary exacerbations and increasing or extending the time between the onset of future pulmonary exacerbations in the high-risk cystic fibrosis patients. In embodiments, the methods to treat the Pseudomonas aeruginosa pulmonary infection further comprise reducing the severity of pulmonary exacerbations in the high-risk cystic fibrosis patients. In embodiments, the Pseudomonas aeruginosa pulmonary infection is a chronic Pseudomonas aeruginosa pulmonary infection. In embodiments, the fluoroquinolone is levofloxacin. In embodiments, the fluoroquinolone is ofloxacin. In embodiments, the high-risk patient experienced at least 3 occurrences of a pulmonary exacerbation in the prior year. In embodiments, the high-risk patient experienced at least 3 occurrences of a pulmonary exacerbation in the prior year. In embodiments, the aerosol of the solution i administered to a patient whose symptoms or pulmonary bacterial infections are not adequately controlled by a different antibiotic therapy.

The disclosure provides methods to treat Staphylococcus aureus pulmonary infections in high-risk cystic fibrosis patients by administering a therapeutically effective amount of an aerosol of a solution comprising a fluoroquinolone. In embodiments, the methods to treat the Staphylococcus aureus pulmonary infection further comprise reducing the incidents of future Staphylococcus aureus pulmonary infections in the high-risk cystic fibrosis patients. In embodiments, the methods to treat the Staphylococcus aureus pulmonary infection further comprise increasing or extending the time between the onset of future Staphylococcus aureus pulmonary infections in the high-risk cystic fibrosis patients. In embodiment, the methods to treat the Staphylococcus aureus pulmonary infection further comprise reducing the incidents of future Staphylococcus aureus pulmonary infections and increasing or extending the time between the onset of future Staphylococcus aureus pulmonary infections in the high-risk cystic fibrosis patients. In embodiments, the methods to treat the Staphylococcus aureus pulmonary infection further comprise reducing the severity of the Staphylococcus aureus pulmonary infections in the high-risk cystic fibrosis patients. In embodiments, the methods to treat the Staphylococcus aureus pulmonary infection further comprise reducing the incidents of future pulmonary exacerbations in the high-risk cystic fibrosis patients. In embodiments, the methods to treat the Staphylococcus aureus pulmonary infection further comprise increasing or extending the time between the onset of future pulmonary exacerbations in the high-risk cystic fibrosis patients. In embodiment, the methods to treat the Staphylococcus aureus pulmonary infection further comprise reducing the incidents of future pulmonary exacerbations and increasing or extending the time between the onset of future pulmonary exacerbations in the high-risk cystic fibrosis patients. In embodiments, the methods to treat the Staphylococcus aureus pulmonary infection further comprise reducing the severity of pulmonary exacerbations in the high-risk cystic fibrosis patients. In embodiments, the Staphylococcus aureus pulmonary infection is a chronic Staphylococcus aureus pulmonary infection. In embodiments, the fluoroquinolone is levofloxacin. In embodiments, the fluoroquinolone is ofloxacin. In embodiments, the high-risk patient experienced at least 3 occurrences of a pulmonary exacerbation in the prior year. In embodiments, the high-risk patient experienced at least 3 occurrences of a pulmonary exacerbation in the prior year. In embodiments, the aerosol of the solution i administered to a patient whose symptoms or pulmonary bacterial infections are not adequately controlled by a different antibiotic therapy.

The disclosure provides methods to reduce the incidents of pulmonary exacerbations in high-risk cystic fibrosis patients by administering a therapeutically effective amount of an aerosol of a solution comprising a fluoroquinolone. In embodiments, the fluoroquinolone is levofloxacin. In embodiments, the fluoroquinolone is ofloxacin. In embodiments, the cystic fibrosis patient has a pulmonary bacterial infection, such as a Pseudomonas aeruginosa pulmonary infection. In embodiments, the high-risk patient experienced at least 3 occurrences of a pulmonary exacerbation in the prior year.

The disclosure provides methods to extend the time between the onset of pulmonary exacerbations in high-risk cystic fibrosis patients by administering a therapeutically effective amount of an aerosol of a solution comprising a fluoroquinolone. In embodiments, the fluoroquinolone is levofloxacin. In embodiments, the fluoroquinolone is ofloxacin. In embodiments, the cystic fibrosis patient has a pulmonary bacterial infection, such as a Pseudomonas aeruginosa pulmonary infection. In embodiments, the high-risk patient experienced at least 3 occurrences of a pulmonary exacerbation in the prior year.

The disclosure provides methods to reduce the severity of pulmonary exacerbations in high-risk cystic fibrosis patients by administering a therapeutically effective amount of an aerosol of a solution comprising a fluoroquinolone. In embodiments, the fluoroquinolone is levofloxacin. In embodiments, the fluoroquinolone is ofloxacin. In embodiments, the cystic fibrosis patient has a pulmonary bacterial infection, such as a Pseudomonas aeruginosa pulmonary infection. In embodiments, the high-risk patient experienced at least 3 occurrences of a pulmonary exacerbation in the prior year.

The disclosure provides methods to achieve one or more of the following: (a) reduce the incidents of pulmonary exacerbations in high-risk cystic fibrosis patients; (b) extend the time between the onset of pulmonary exacerbations in high-risk cystic fibrosis patients, and (c) reduce the severity of pulmonary exacerbations in high-risk cystic fibrosis patients by administering a therapeutically effective amount of an aerosol of a solution comprising a fluoroquinolone. In embodiments, the fluoroquinolone is levofloxacin. In embodiments, the fluoroquinolone is ofloxacin. In embodiments, the cystic fibrosis patient has a pulmonary bacterial infection, such as a Pseudomonas aeruginosa pulmonary infection. In embodiments, the high-risk patient experienced at least 3 occurrences of a pulmonary exacerbation in the prior year.

The disclosure provides methods to reduce a Pseudomonas aeruginosa sputum density in high-risk cystic fibrosis patients by administering a therapeutically effective amount of an aerosol of a solution comprising a fluoroquinolone. In embodiments, the fluoroquinolone is levofloxacin. In embodiments, the fluoroquinolone is ofloxacin. In embodiments, the high-risk patient experienced at least 3 occurrences of a pulmonary exacerbation in the prior year.

In other embodiments of the methods described herein, the high-risk cystic fibrosis patient has, at the time of the pulmonary exacerbation (e.g., diagnosis for treatment of a pulmonary bacterial infection), a forced expiratory volume in 1 second (FEV₁) between 10% and 95% or between 25% and 85% of their predicted values using Hankinson/NHANES III reference equations. In embodiments, the high risk cystic fibrosis patient has a forced expiratory volume in 1 second (FEV₁) of 10% to 55%; of 55% to 85%; of 60% to 85%; or greater than 75% of the predicted value using Hankinson/NHANES III reference equations at the time of the pulmonary exacerbation. The Hankinson/NHANES III reference equations are well-known in the art, and described in Hankinson et al, Am J Respir Crit Care Med, 159:179-187 (1999).

In other embodiments of the methods described herein, the high-risk cystic fibrosis patient has a chronic airway infection with Pseudomonas aeruginosa and received at least three 28-day courses of an inhaled antibiotic in the prior year (e.g., an inhaled aminoglycoside formulation; an inhaled monobactam formulation; an inhaled polymyxin formulation; or a combination of two or more thereof). In embodiments, the high-risk cystic fibrosis patient has a chronic airway infection with Pseudomonas aeruginosa and had received at least three 28-day courses of an inhaled tobramycin formulation in the prior year. Inhaled tobramycin formulations are known in the art and are commercially available, such as TOBI® (Novartis, East Hanover, N.J.) and BETHKIS® (Chiesi, Cary, N.C.).

In other embodiments of the methods described herein, the high-risk cystic fibrosis patient had at least three 28-day courses of intravenous antibiotic treatments for a pulmonary Pseudomonas aeruginosa infection in the prior year. In other embodiments of the methods described herein, the high-risk cystic fibrosis patient had at least three 28-day courses of intravenous antibiotic treatments for a pulmonary bacterial infection in the prior year. The intravenous antibiotic can be any known in the art, including, for example, a beta-lactam antibiotic or an aminoglycoside antibiotic.

Inhalation Solutions

For purposes of the method described herein, a fluoroquinolone inhalation solution, levofloxacin inhalation solution, and ofloxacin inhalation solution may be administered using an inhaler. In some embodiments, a fluoroquinolone (including levofloxacin and ofloxacin) is produced as a pharmaceutical composition suitable for aerosol formation, good taste, storage stability, and patient safety and tolerability. In some embodiments, the isoform content of the manufactured fluoroquinolone may be optimized for tolerability, antimicrobial activity and stability.

The inhalation solutions can include a divalent or trivalent cation. The divalent or trivalent cation can include, for example, magnesium, calcium, zinc, copper, aluminum, and iron. In some embodiments, the solution comprises magnesium chloride, magnesium sulfate, zinc chloride, or copper chloride. In some embodiments, the solution comprises magnesium chloride. In some embodiments, the divalent or trivalent cation concentration can be from about 25 mM to about 400 mM, from about 50 mM to about 400 mM, from about 100 mM to about 300 mM, from about 100 mM to about 250 mM, from about 125 mM to about 250 mM, from about 150 mM to about 250 mM, from about 175 mM to about 225 mM, from about 180 mM to about 220 mM, and from about 190 mM to about 210 mM. In some embodiments, the concentration is about 200 mM. In some embodiments, the magnesium chloride, magnesium sulfate, zinc chloride, or copper chloride can have a concentration from about 5% to about 25%, from about 10% to about 20%, and from about 15% to about 20%. In some embodiments, the ratio of fluoroquinolone to divalent or trivalent cation can be 1:1 to 2:1 or 1:1 to 1:2.

The inhalation solution can have a fluoroquinolone concentration, for example, levofloxacin or ofloxacin, greater than about 50 mg/ml, about 60 mg/ml, about 70 mg/ml, about 80 mg/ml, about 90 mg/ml, about 100 mg/ml, about 110 mg/ml, about 120 mg/ml, about 130 mg/ml, about 140 mg/ml, about 150 mg/ml, about 160 mg/ml, about 170 mg/ml, about 180 mg/ml, about 190 mg/ml, and about 200 mg/ml. In some embodiments, the inhalation solution can have a fluoroquinolone concentration, for example, levofloxacin or ofloxacin, from about 50 mg/ml to about 200 mg/ml, from about 75 mg/ml to about 150 mg/ml, from about 80 mg/ml to about 125 mg/ml, from about 80 mg/ml to about 120 mg/ml, from about 90 mg/ml to about 125 mg/ml, from about 90 mg/ml to about 120 mg/ml, and from about 90 mg/ml to about 110 mg/ml. In some embodiments, the concentration is about 100 mg/ml.

The inhalation solution can have an osmolality from about 300 mOsmol/kg to about 500 mOsmol/kg, from about 325 mOsmol/kg to about 450 mOsmol/kg, from about 350 mOsmol/kg to about 425 mOsmol/kg, and from about 350 mOsmol/kg to about 400 mOsmol/kg. In some embodiments, the osmolality of the inhalation solution is greater than about 300 mOsmol/kg, about 325 mOsmol/kg, about 350 mOsmol/kg, about 375 mOsmol/kg, about 400 mOsmol/kg, about 425 mOsmol/kg, about 450 mOsmol/kg, about 475 mOsmol/kg, and about 500 mOsmol/kg.

The inhalation solution can have a pH from about 4.5 to about 8.5, from about 5.0 to about 8.0, from about 5.0 to about 7.0, from about 5.0 to about 6.5, from about 5.5 to about 6.5, and from 6.0 to about 6.5.

The inhalation solution can optionally comprise a conventional pharmaceutical carrier, excipient or the like (e.g., mannitol, lactose, starch, magnesium stearate, sodium saccharine, talcum, cellulose, sodium crosscarmellose, glucose, gelatin, sucrose, magnesium carbonate, and the like), or auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate, and the like). In some embodiments, the inhalation solution can lack a conventional pharmaceutical carrier, excipient or the like. Some embodiments include a formulation lacking lactose. Some embodiments comprise lactose at a concentration less than about 10%, 5%, 1%, or 0.1%. In some embodiments, the inhalation solution can consist essentially of levofloxacin or ofloxacin and a divalent or trivalent cation.

In some embodiments, an inhalation solution can comprise a levofloxacin concentration between about 75 mg/ml to about 150 mg/ml, a magnesium chloride concentration between about 150 mM to about 250 mM, a pH between about 5 to about 7; an osmolality of between about 300 mOsmol/kg to about 600 mOsmol/kg, and lacks lactose.

In some embodiments, an inhalation solution comprises a levofloxacin concentration of about 100 mg/ml, a magnesium chloride concentration of about 200 mM, a pH of about 6.2, an osmolality of about 383 mOsmol/kg. In some embodiments, a formulation consists essentially of a levofloxacin concentration of about 90 mg/ml to about 110 mg/ml, a magnesium chloride concentration of about 180 mM to about 220 mM, a pH of about 5 to about 7, an osmolality of about 300 mOsmol/kg to 500 mOsmol/kg.

The amount of active compound administered will be dependent on the subject and disease state being treated, the severity of the affliction, the manner and schedule of administration and the judgment of the prescribing physician; for example, a likely dose range for aerosol administration of levofloxacin would be about 20 to 300 mg per day, the active agents being selected for longer or shorter pulmonary half-lives, respectively. In some embodiments, a likely dose range for aerosol administration of levofloxacin would be about 100 to 300 mg twice daily.

Administration of the fluoroquinolone antimicrobial agents disclosed herein or the pharmaceutically acceptable salts thereof can be via any of the accepted modes of administration for agents that serve similar utilities including, but not limited to, aerosol inhalation. Methods, devices and compositions for delivery are described in U.S. Pat. No. 7,838,532, incorporated by reference in its entirety.

Aerosol Delivery of Inhalation Solutions

For pulmonary administration, the upper airways are avoided in favor of the middle and lower airways. Pulmonary drug delivery may be accomplished by inhalation of an aerosol through the mouth and throat. Particles having a mass median aerodynamic diameter (MMAD) of greater than about 5 microns generally do not reach the lung; instead, they tend to impact the back of the throat and are swallowed and possibly orally absorbed. Particles having diameters of about 2 to about 5 microns are small enough to reach the upper- to mid-pulmonary region (conducting airways), but are too large to reach the alveoli. Smaller particles, i.e., about 0.5 to about 2 microns, are capable of reaching the alveolar region. Particles having diameters smaller than about 0.5 microns can also be deposited in the alveolar region by sedimentation, although very small particles may be exhaled.

In embodiments, a nebulizer is selected on the basis of allowing the aerosol of a solution of a fluoroquinolone disclosed herein having an MMAD predominantly between about 2 to about 5 microns. In embodiments, the delivered amount of fluoroquinolone provides a therapeutic effect for pulmonary infections. The nebulizer can deliver an aerosol comprising a mass median aerodynamic diameter from about 2 microns to about 5 microns with a geometric standard deviation less than or equal to about 2.5 microns, a mass median aerodynamic diameter from about 2.5 microns to about 4.5 microns with a geometric standard deviation less than or equal to about 1.8 microns, and a mass median aerodynamic diameter from about 2.8 microns to about 4.3 microns with a geometric standard deviation less than or equal to about 2 microns. In some embodiments, the aerosol can be produced using a vibrating mesh nebulizer. An example of a vibrating mesh nebulizer includes the PARI E-FLOW® nebulizer or a nebulizer using PARI eFlow technology. More examples of nebulizers are provided in U.S. Pat. Nos. 4,268,460; 4,253,468; 4,046,146; 3,826,255; 4,649,911; 4,510,929; 4,624,251; 5,164,740; 5,586,550; 5,758,637; 6,644,304; 6,338,443; 5,906,202; 5,934,272; 5,960,792; 5,971,951; 6,070,575; 6,192,876; 6,230,706; 6,349,719; 6,367,470; 6,543,442; 6,584,971; 6,601,581; 4,263,907; 5,709,202; 5,823,179; 6,192,876; 6,644,304; 5,549,102; 6,083,922; 6,161,536; 6,264,922; 6,557,549; and 6,612,303 all of which are hereby incorporated by reference in their entireties. More commercial examples of nebulizers that can be used with the formulations described herein include Respirgard II®, Aeroneb®, Aeroneb® Pro, and Aeroneb® Go produced by Aerogen; AERx® and AERx Essence® produced by Aradigm; Porta-Neb®, Freeway Freedom®, Sidestream, Ventstream and I-neb produced by Respironics, Inc.; and PARI LC-Plus® PARI LC-Start, produced by PARI, GmbH. By further non-limiting example, U.S. Pat. No. 6,196,219, is hereby incorporated by reference in its entirety.

“Respirable delivered dose” or “RDD” is the dose or amount of drug delivered to the lung of a patient using a nebulizer or other aerosol delivery device. The RDD is estimated from the inspiratory phase of a breath simulation device programmed to the European Standard pattern of 15 breaths per minute, with an inspiration to expiration ratio of 1:1, and measurement of particles emitted from a nebulizer with a size of about 5 microns or less. The amount of levofloxacin or ofloxacin that can be administered to the lungs with an aerosol dose, such as the RDD, can include at least about 20 mg, about 30 mg, about 40 mg, about 50 mg, about 60 mg, about 70 mg, about 80 mg, about 90 mg, about 100 mg, about 110 mg, about 120 mg, about 125 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, and about 800 mg. In some embodiments, the amount of levofloxacin or ofloxacin that can be administered to the lungs with an aerosol dose, such as a respirable drug dose (RDD), that can include about 20 mg to about 500 mg; about 50 mg to about 400 mg; or about 50 mg to about 300 mg.

The aerosol can be administered to the lungs in less than about 10 minutes, about 5 minutes, about 4 minutes, about 3 minutes, about 2 minutes, and about 1 minute.

Bacterial Infections

Methods and inhalation solutions described herein can be used to treat pulmonary infections and disorders. Examples of such disorders can include cystic fibrosis, pneumonia, and chronic obstructive pulmonary disease, including chronic bronchitis, and some asthmas. In embodiments, the disorder is cystic fibrosis. Some embodiments include treating an infection comprising one or more bacteria selected from the group consisting of Pseudomonas aeruginosa, Pseudomonas fluorescens, Pseudomonas acidovorans, Pseudomonas alcaligenes, Pseudomonas putida, Stenotrophomonas maltophilia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Morganella morganii, Proteus mirabilis, Proteus vulgaris, Providencia alcalifaciens, Providencia rettgeri, Providencia stuartii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Helicobacter pylori, Campylobacter fetus, Campylobacter jejuni, Campylobacter coli, Borrelia burgdorferi, Vibrio cholera, Vibrio parahaemolyticus, Legionella pneumophila, Listeria monocytogenes, Neisseria gonorrhoeae, Neisseria meningitidis, Burkholderia cepacia, Francisella tularensis, Kingella, and Moraxella. In embodiments, the infection is a Pseudomonas infection. In embodiments, the infection is Pseudomonas aeruginosa, Pseudomonas fluorescens, Pseudomonas acidovorans, Pseudomonas alcaligenes, or Pseudomonas putida. In embodiments, the infection is Pseudomonas aeruginosa.

In embodiments, the lung infection is caused by a gram-negative anaerobic bacteria. In embodiments, the lung infection comprises one or more of the bacteria selected from the group consisting of Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, and Bacteroides splanchnicus. In some embodiments, the lung infection is caused by a gram-positive bacteria. In some embodiments, the lung infection comprises one or more of the bacteria selected from the group consisting of Corynebacterium diphtheriae, Corynebacterium ulcerans, Streptococcus pneumoniae, Streptococcus agalactiae, Streptococcus pyogenes, Streptococcus milleri; Streptococcus (Group G); Streptococcus (Group C/F); Enterococcus faecalis, and Enterococcus faecium.

In embodiments, the lung infection is caused by a Staphylococcus bacteria. In embodiments, the Staphylococcus bacteria is Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus. In embodiments, the Staphylococcus bacteria is Staphylococcus aureus.

In embodiments, the lung infection is caused by a gram-positive anaerobic bacteria. In some embodiments, the lung infection is caused by one or more bacteria selected from the group consisting of Clostridium difficile, Clostridium perfringens, Clostridium tetini, and Clostridium botulinum.

In embodiments, the lung infection is caused by an acid-fast bacteria. In some embodiments, the lung infection is caused by one or more bacteria selected from the group consisting of Mycobacterium tuberculosis, Mycobacterium avium, Mycobacterium intracellulare, and Mycobacterium leprae. In embodiments, the lung infection is caused by an atypical bacteria. In embodiments, the lung infection is caused by one or more bacteria selected from the group consisting of Chlamydia pneumoniae and Mycoplasma pneumoniae.

### EMBODIMENTS

The disclosure includes, but is not limited to, the following embodiments.

Embodiment 1. A method for treating a Pseudomonas aeruginosa pulmonary infection in a high-risk cystic fibrosis patient in need thereof comprising administering a therapeutically effective amount of an aerosol of a solution comprising levofloxacin to the high-risk cystic fibrosis patient.

Embodiment 2. A method for reducing the incidents of a pulmonary exacerbation in a high-risk cystic fibrosis patient in need thereof comprising administering a therapeutically effective amount of an aerosol of a solution comprising levofloxacin to the high-risk cystic fibrosis patient.

Embodiment 3. A method for extending the time between the onset of pulmonary exacerbations in a high-risk cystic fibrosis patient in need thereof comprising administering a therapeutically effective amount of an aerosol of a solution comprising levofloxacin to the high-risk cystic fibrosis patient.

Embodiment 4. A method for treating a pulmonary bacterial infection in a high-risk cystic fibrosis patient in need thereof comprising administering a therapeutically effective amount of an aerosol of a solution comprising levofloxacin to the high-risk cystic fibrosis patient

Embodiment 5. A method of treating a pulmonary exacerbation in a high-risk cystic fibrosis patient in need thereof comprising administering a therapeutically effective amount of an aerosol of a solution comprising levofloxacin to the high-risk cystic fibrosis patient

Embodiment 6. A method of treating cystic fibrosis in a high-risk cystic fibrosis patient in need thereof comprising administering a therapeutically effective amount of an aerosol of a solution comprising levofloxacin to the high-risk cystic fibrosis patient.

Embodiment 7. A method of reducing a Pseudomonas aeruginosa sputum density in a high-risk cystic fibrosis patient in need thereof comprising administering a therapeutically effective amount of an aerosol of a solution comprising levofloxacin to the high-risk cystic fibrosis patient.

Embodiment 8. The method of any one of Embodiments 1-7, wherein the high-risk patient had at least 3 occurrences of a pulmonary exacerbation in the prior year.

Embodiment 9. The method of any one of Embodiments 1-7, wherein the high-risk patient had at least 4 occurrences of a pulmonary exacerbation in the prior year.

Embodiment 10. The method of any one of Embodiments 1-7, wherein the high-risk patient had at least 5 occurrences of a pulmonary exacerbation in the prior year.

Embodiment 11. The method of any one of Embodiments 2, 3, 5, and 8-10, wherein the pulmonary exacerbation comprises at least four of the following: (i) a change in sputum; (ii) a new or increased hemoptysis; (iii) an increased cough; (iv) increased dyspnea; (v) malaise, fatigue, or lethargy; (vi) a temperature above 38° C.; (vii) anorexia or weight loss; (viii) sinus pain or tenderness; (ix) a change in sinus discharge; (x) a change in physical examination of the chest; (xi) a decrease in pulmonary function by 10% or more from a previously recorded value; and (xii) a radiographic change indicative of pulmonary infection.

Embodiment 12. The method of any one of Embodiments 2, 3, 5, and 8-10, wherein the pulmonary exacerbation is a pulmonary bacterial infection

Embodiment 13. The method of any one of Embodiments 2, 3, 5, and 8-10, wherein the pulmonary exacerbation is a Pseudomonas aeruginosa pulmonary infection

Embodiment 14. The method of any one of Embodiments 2, 3, 5, and 8-10, wherein the pulmonary exacerbation is a Pseudomonas pulmonary infection

Embodiment 15. The method of any one of Embodiments 2, 3, 5, and 8-10, wherein the pulmonary exacerbation is a Staphylococcus aureus pulmonary infection.

Embodiment 16. The method of any one of Embodiments 2, 3, 5, and 8-10, wherein the pulmonary exacerbation is a Staphylococcus pulmonary infection.

Embodiment 17. The method of any one of Embodiments 2, 3, 5, and 8-10, wherein the pulmonary exacerbation is a decrease of 10% or more in the FEV₁ of the predicted value using Hankinson/NHANES III reference equation.

Embodiment 18. The method of any one of Embodiments 1-17, wherein the high-risk patient has a forced expiratory volume in 1 second (FEV₁) between 25 and 85 percent of their predicted values using Hankinson/NHANES III reference equation.

Embodiment 19. The method of any one of Embodiments 1-17, wherein the high-risk patient had a chronic airway infection with Pseudomonas aeruginosa and had received at least three 28-day courses of an inhaled tobramycin formulation in the prior year.

Embodiment 20. The method of any one of Embodiments 1-17, wherein the high-risk patient had a chronic airway infection with Pseudomonas aeruginosa and had received at least three 28-day courses of an inhaled aminoglycoside formulation in the prior year, an inhaled monobactam formulation in the prior year, an inhaled polymyxin formulation in the prior year, or a combination of two or more thereof.

Embodiment 21. The method of any one of Embodiments 1-20, wherein the solution comprises levofloxacin or ofloxacin and a divalent or trivalent cation.

Embodiment 22. The method of Embodiment 21, wherein the trivalent cation is aluminum or iron.

Embodiment 23. The method of any one of Embodiments 1-20, wherein the solution comprises levofloxacin and a divalent cation.

Embodiment 24. The method of Embodiment 23, wherein the divalent cation is magnesium, calcium, zinc, copper, or iron.

Embodiment 25. The method of Embodiment 23, wherein the divalent cation is magnesium.

Embodiment 26. The method of any one of Embodiments 1-20, wherein the solution comprises about 75 mg/ml to about 150 mg/ml of levofloxacin and about 150 mM to about 250 mM of a divalent cation.

Embodiment 27. The method of Embodiment 26, wherein the divalent cation is magnesium, calcium, zinc, copper, or iron.

Embodiment 28. The method of Embodiment 26, wherein the divalent cation is magnesium.

Embodiment 29. The method of any one of Embodiments 1-20, wherein the solution comprises about 90 mg/ml to about 110 mg/ml of levofloxacin and about 190 mM to about 210 mM of a divalent cation.

Embodiment 30. The method of Embodiment 29, wherein the divalent cation is magnesium, calcium, zinc, copper, or iron.

Embodiment 31. The method of Embodiment 29, wherein the divalent cation is magnesium.

Embodiment 32. The method of any one of Embodiments 1-20, wherein the solution comprises about 80 mg/ml to about 120 mg/ml of levofloxacin and about 180 mM to about 220 mM of a divalent cation.

Embodiment 33. The method of Embodiment 32, wherein the divalent cation is magnesium, calcium, zinc, copper, or iron.

Embodiment 34. The method of Embodiment 32, wherein the divalent cation is magnesium.

Embodiment 35. The method of any one of Embodiments 1-20, wherein solution comprises about 100 mg/ml of levofloxacin and about 200 mM magnesium chloride.

Embodiment 36. A method for treating a Pseudomonas aeruginosa pulmonary infection in a high-risk cystic fibrosis patient in need thereof comprising administering a therapeutically effective amount of an aerosol of a solution comprising ofloxacin to the high-risk cystic fibrosis patient.

Embodiment 37. A method for reducing the incidents of a pulmonary exacerbation in a high-risk cystic fibrosis patient in need thereof comprising administering a therapeutically effective amount of an aerosol of a solution comprising ofloxacin to the high-risk cystic fibrosis patient.

Embodiment 38. A method for extending the time between the onset of pulmonary exacerbations in a high-risk cystic fibrosis patient in need thereof comprising administering a therapeutically effective amount of an aerosol of a solution comprising ofloxacin to the high-risk cystic fibrosis patient.

Embodiment 39. A method for treating a pulmonary bacterial infection in a high-risk cystic fibrosis patient in need thereof comprising administering a therapeutically effective amount of an aerosol of a solution comprising ofloxacin to the high-risk cystic fibrosis patient.

Embodiment 40. A method of treating a pulmonary exacerbation in a high-risk cystic fibrosis patient in need thereof comprising administering a therapeutically effective amount of an aerosol of a solution comprising ofloxacin to the high-risk cystic fibrosis patient.

Embodiment 41. A method of treating cystic fibrosis in a high-risk cystic fibrosis patient in need thereof comprising administering a therapeutically effective amount of an aerosol of a solution comprising ofloxacin to the high-risk cystic fibrosis patient.

Embodiment 42. A method of reducing a Pseudomonas aeruginosa sputum density in a high-risk cystic fibrosis patient in need thereof comprising administering a therapeutically effective amount of an aerosol of a solution comprising ofloxacin to the high-risk cystic fibrosis patient.

Embodiment 43. The method of any one of Embodiments 36-42, wherein the high-risk patient had at least 3 occurrences of a pulmonary exacerbation in the prior year.

Embodiment 44. A method for treating a Pseudomonas aeruginosa pulmonary infection in a high-risk cystic fibrosis patient in need thereof comprising administering a therapeutically effective amount of an aerosol of a solution comprising a fluoroquinolone to the high-risk cystic fibrosis patient.

Embodiment 45. A method for reducing the incidents of a pulmonary exacerbation in a high-risk cystic fibrosis patient in need thereof comprising administering a therapeutically effective amount of an aerosol of a solution comprising a fluoroquinolone to the high-risk cystic fibrosis patient.

Embodiment 46. A method for extending the time between the onset of pulmonary exacerbations in a high-risk cystic fibrosis patient in need thereof comprising administering a therapeutically effective amount of an aerosol of a solution comprising a fluoroquinolone to the high-risk cystic fibrosis patient.

Embodiment 47. A method of reducing a Pseudomonas aeruginosa sputum density in a high-risk cystic fibrosis patient in need thereof comprising administering a therapeutically effective amount of an aerosol of a solution comprising a fluoroquinolone to the high-risk cystic fibrosis patient.

Embodiment 48. A method for treating a pulmonary bacterial infection in a high-risk cystic fibrosis patient in need thereof comprising administering a therapeutically effective amount of an aerosol of a solution comprising a fluoroquinolone to the high-risk cystic fibrosis patient.

Embodiment 49. A method of treating a pulmonary exacerbation in a high-risk cystic fibrosis patient in need thereof comprising administering a therapeutically effective amount of an aerosol of a solution comprising a fluoroquinolone to the high-risk cystic fibrosis patient.

Embodiment 50. A method of treating cystic fibrosis in a high-risk cystic fibrosis patient in need thereof comprising administering a therapeutically effective amount of an aerosol of a solution comprising a fluoroquinolone to the high-risk cystic fibrosis patient.

Embodiment 51. The method of anyone of Embodiments 44-50, wherein the fluoroquinolone is ciprofloxacin, enoxacin, gatifloxacin, gemifloxacin, lomefloxacin, moxifloxacin, norfloxacin, pefloxacin, sparfloxacin, garenoxacin, sitagloxacin, or DX-619.

Embodiment 52. The method of anyone of Embodiments 44-50, wherein the high-risk patient had at least 3 occurrences of a pulmonary exacerbation in the prior year.

Embodiment 53. A method for treating a Pseudomonas aeruginosa pulmonary infection in a high-risk cystic fibrosis patient in need thereof comprising administering by inhalation a therapeutically effective amount of a pharmaceutical composition comprising an antibiotic to the high-risk cystic fibrosis patient.

Embodiment 54. A method for reducing the incidents of a pulmonary exacerbation in a high-risk cystic fibrosis patient in need thereof comprising administering by inhalation a therapeutically effective amount of a pharmaceutical composition comprising an antibiotic to the high-risk cystic fibrosis patient.

Embodiment 55. A method for extending the time between the onset of pulmonary exacerbations in a high-risk cystic fibrosis patient in need thereof comprising administering by inhalation a therapeutically effective amount of a pharmaceutical composition comprising an antibiotic to the high-risk cystic fibrosis patient.

Embodiment 56. A method for treating a pulmonary bacterial infection in a high-risk cystic fibrosis patient in need thereof comprising administering by inhalation a therapeutically effective amount of a pharmaceutical composition comprising an antibiotic to the high-risk cystic fibrosis patient.

Embodiment 57. A method of treating a pulmonary exacerbation in a high-risk cystic fibrosis patient in need thereof comprising administering by inhalation a therapeutically effective amount of a pharmaceutical composition comprising an antibiotic to the high-risk cystic fibrosis patient.

Embodiment 58. A method of treating cystic fibrosis in a high-risk cystic fibrosis patient in need thereof comprising administering by inhalation a therapeutically effective amount of a pharmaceutical composition comprising an antibiotic to the high-risk cystic fibrosis patient.

Embodiment 59. The method of any one of Embodiments 53-58, wherein the high-risk patient had at least 3 occurrences of a pulmonary exacerbation in the prior year.

Embodiment 60. The method of any one of Embodiments 53-59, wherein the antibiotic is tobramycin.

Embodiment 61. The method of any one of Embodiments 53-59, wherein the antibiotic is aztreonam.

Embodiment 62. The method of Embodiment 61, wherein the aztreonam is aztreonam lysine.

### EXAMPLES

The following examples are for purposes of illustration only and are not intended to limit the spirit or scope of the disclosure or claims.

### Example 1

Pulmonary exacerbations are important events among persons with cystic fibrosis and are a significant risk factor for future pulmonary exacerbations and pulmonary deterioration. Levofloxacin inhalation solution was evaluated in an open-label study with tobramycin inhalation solution over three 28-day cycles, with intervals of 28-days off treatment. Here we describe the proportion of patients who experienced pulmonary exacerbations and who were treated with antibiotics stratified by the number of clinician-reported pulmonary exacerbations patients had experienced in the prior year (prior-year pulmonary exacerbations). In this example “levofloxacin inhalation solution” refers to an aerosol of a solution containing about 100 mg/ml of levofloxacin and about 200 mM magnesium chloride.

In this example, a pulmonary exacerbation occurred when a patient met at least four of the twelve Fuchs criteria and was prescribed an antibiotic treatment. Prior-year pulmonary exacerbation categories were defined as 0, 1-2, or ≥3 pulmonary exacerbations. The incidents of pulmonary exacerbations during the study was compared between levofloxacin inhalation solution (N=189) and tobramycin inhalation solution (N=93) treatment groups, both overall and among prior-year exacerbation subgroups.

Baseline demographics, including mean FEV₁% predicted, were similar between treatment groups and across prior-year exacerbation categories. The overall exacerbation incidents was not substantially different between levofloxacin inhalation solution patients (53/189=28.0% [95% CI 21.8%, 35.0%]) and tobramycin inhalation solution patients (34/93=36.6% [26.8%, 47.2%]. Incidence of pulmonary exacerbations increased with increasing prior-year pulmonary exacerbations, but did not differ between treatment groups with less than 3 prior-year pulmonary exacerbations. For those with three or more prior-year pulmonary exacerbations, 17/59 levofloxacin inhalation solution patients (28.8% [17.8%, 42.1%]) had a pulmonary exacerbation compared to 14/25 tobramycin inhalation solution patients (56.0% [34.9%, 75.6%]). This 27.2% difference in pulmonary exacerbation incidents was statistically significant (P=0.03; two-tailed Fisher's exact test).

The study unexpectedly showed that patients with three or more pulmonary exacerbations in the prior year had the greatest pulmonary exacerbation-reduction benefit from levofloxacin inhalation solution treatment.

### Example 2

This was a randomized, double-blind, placebo-controlled trial. Eligible patients were randomized 2:1 to receive 28 days of treatment with either levofloxacin inhalation solution 240 mg (2.4 mL of a 100 mg/ml of levofloxacin and magnesium chloride) twice daily or a 0.9% saline-based placebo (color matched with riboflavin), with a 28-day follow-up period off therapy. Levofloxacin inhalation solution or placebo was delivered with a PARI investigational eFlow® nebulizer.

Participants. Eligible patients were ≥12 years of age with a documented cystic fibrosis diagnosis, a forced expiratory volume in 1 second (FEV₁) between 25 and 85 percent of their predicted values using Hankinson/NHANES III reference equations, chronic airways infection with Pseudomonas aeruginosa, and had received at least three 28-day courses (>84 days) of inhaled tobramycin inhalation solution over the year prior to screening. Chronic Pseudomonas aeruginosa infection was defined as report of a respiratory secretion culture positive for Pseudomonas aeruginosa in the year immediately prior to screening and a positive culture obtained at the screening visit. Patients continued their routine respiratory care and medications during the study. Patients were not permitted to use other antipseudomonal antimicrobials other than Study Drug unless deemed necessary by the Investigator to treat a suspected exacerbation.

Endpoints. The primary efficacy endpoint was the time to a pulmonary exacerbation of cystic fibrosis lung disease (i.e., pulmonary illness). To meet the definition of a pulmonary exacerbation, patients must have had changes in ≥4 of the 12 Fuchs criteria, independent of a clinician's decision to treat the patient with an antibiotic. Use of the term “pulmonary exacerbation” in this manner is a modification of the above definition, which requires a clinician's decision to affirmatively treat with antibiotics (e.g. IV antibiotics) for a respiratory (as opposed to other) event. Accordingly, in this example, the term “modified pulmonary exacerbation endpoint” or “modified pulmonary exacerbation” or “protocol-defined pulmonary exacerbation” is used because the clinician may or may not have made a decision to treat a patient with an antibiotic when the patient had at least 4 of the 12 Fuchs criteria. In addition to those patients meeting this modified pulmonary exacerbation endpoint, patients were considered to have experienced a pulmonary exacerbation if they discontinued from the study early for any reason, died, or received an antipseudomonal antimicrobial agent for an event that did not meet the predefined criteria but was determined to be a pulmonary exacerbation for the purposes of the primary endpoint by an independent, blinded, exacerbation adjudication committee. The adjudication committee reviewed all instances in which patients received additional antipseudomonal antibiotics but did not meet the protocol definition of a pulmonary exacerbation to determine if these treatments were associated with a pulmonary exacerbation.

Additional endpoints included absolute change from baseline in FEV₁ percent predicted, change from baseline in CF Questionnaire-Revised (CFQ-R) respiratory symptom score, and change from baseline in sputum Pseudomonas aeruginosa density (log₁₀ colony-forming units (CFU) per gram sputum). Quittner et al, Chest, 128:2347-2354 (2005). Changes in respiratory signs and symptoms were systematically collected using the Respiratory Signs and Symptoms Questionnaire (RSSQ). Konstan et al, J Cyst Fibros, 13:148-155 (2014). Adverse events and serious adverse events were captured from baseline to the final visit for each patient. In addition to standard adverse event reporting, all worsening of Fuchs criteria, as captured on the RSSQ, were captured as adverse events.

Respiratory secretions (throat swabs or sputum) were collected at all study visits for selective bacterial culture by central laboratories. Distinct Pseudomonas aeruginosa morphotypes from patients were analyzed separately. Bacterial densities in sputum specimens were determined by dilution plating.

Statistical analysis was performed on the intention to treat (ITT) population consisting of all randomized patients. A hierarchical testing procedure was employed; all tests conducted subsequent to one in which statistical significance was not demonstrated were to be considered as exploratory. The primary efficacy analysis compared the distributions of the time to a pulmonary exacerbation in the treatment groups using a 2-sided stratified (geographic region [US, non-US], age [12 to 18 years, >18 years], and FEV₁ percent predicted at Baseline [55%, ≥55%]) log rank test at the 5% level of significance. The time-to-event distributions in the groups were summarized using the Kaplan-Meier method. Based on the hierarchical testing procedure, if the primary efficacy endpoint did not show a statistically significant difference, the treatment comparisons for the key secondary endpoints were to be considered as exploratory; hence the term “statistical significance” would refer to nominal significance only.

Secondary analyses of change in FEV₁ percent predicted (both absolute and relative changes), change in Pseudomonas aeruginosa sputum density (log₁₀ CFU/g sputum), and change in the Respiratory Domain of the CFQ-R from Baseline to Day 28 were each compared between treatment groups using linear mixed models for repeated measurements that included terms for treatment group (levofloxacin inhalation solution, placebo), visit (Day 14, Day 28), treatment-by-visit interaction, geographic region (US, non-US), age (12 to 18 years, >18 years), and FEV₁ percent predicted at Baseline (<55%, ≥55%). Additional terms included were baseline Pseudomonas aeruginosa sputum density for change in Pseudomonas aeruginosa sputum density and Baseline score for change in the Respiratory Domain of the CFQ-R.

A post-hoc analysis of time to pulmonary exacerbation (i.e., as defined by either the modified pulmonary exacerbation endpoint or by administration of antipseudomonal antibiotics) was performed among patient subgroups defined by the number of pulmonary exacerbations treated with intravenous antibiotics they had experienced in the prior year. Hazard ratios (levofloxacin inhalation solution/placebo) and log rank P values were determined by Kaplan-Meier survival analysis.

Due to lack of prior experience with the modified pulmonary exacerbation endpoint, sample sizes were estimated based upon time-to-need for systemic or inhaled antimicrobials observed for placebo patients of a previous levofloxacin inhalation solution study. In that prior study, an event-free rate of 0.50 at Day 56 was observed. For the current study, a 2:1 (levofloxacin inhalation solution: placebo) randomization, a maximum follow-up time of 56 days, and use of a 2-sided log rank test at the 5% level of significance was determined to require 261 patients to obtain 90% power to detect a hazard ratio (HR) of 0.52 (ratio of the risk of use of a systemic or inhaled antimicrobial for a pulmonary exacerbation in the levofloxacin inhalation solution arm to the risk of same for a pulmonary exacerbation in the placebo arm). The sample size was subsequently increased to account for a secondary endpoint of interest, relative change in FEV₁ percent predicted, in keeping with the primary endpoint of a Phase 3, open-label, randomized trial to compare the safety and efficacy of levofloxacin inhalation solution with TIS over 3 consecutive cycles. Elborn et al, Journal of Cystic Fibrosis, 14(4):507-514 (2015). Accordingly, the planned sample size was increased to 330 patients to obtain >90% power to detect an 8.0 percentage point treatment difference in relative change in FEV₁ percent predicted, assuming a 2-sided test at the 5% level of significance, a standard deviation of 20%, and 2:1 randomization to levofloxacin inhalation solution: placebo. Approximately 415 patients were to be screened to enroll approximately 330 patients, assuming a 20% screening failure rate.

Levofloxacin minimum inhibitory concentrations (MIC) were determined using broth dilution reference methods as published by the Clinical Laboratory Standards Institute (CLSI; REF-M100). Changes in the levofloxacin MIC were evaluated as the proportion of patients for which the levofloxacin MIC of their most resistant Pseudomonas aeruginosa isolate changed by >2 fold (the limit of sensitivity of dilution testing) from baseline to the end of the study using a 2-sided Fisher's exact test with a 5% significance level.

Results. Three hundred and thirty patients were randomized in this study; 220 to receive levofloxacin inhalation solution and 110 placebo. Most patients completed the study, 95.5% of levofloxacin inhalation solution and 99.1% of placebo patients. The disposition and the reasons for discontinuing the study were similar between the treatment groups. As shown in Table 1, baseline characteristics of the two groups were generally similar with the exception of prior year pulmonary exacerbations with the treatment group having a greater proportion of subjects with ≥3 pulmonary exacerbations compared to the placebo group (p=0.011). At the randomization visit, Pseudomonas aeruginosa and Staphylococcus aureus were isolated in 96% and 51% of patients, respectively. There were no differences in baseline Pseudomonas aeruginosa antibiotic susceptibility patterns between the two groups. Concomitant medications were also similar between the two groups at baseline. The median number of inhaled antibiotic courses during the previous year was 6, and 58.7% of the enrolled patients had received 6 or more courses.

TABLE 1
Demographics at Baseline

| | |
|---|---|
| Placebo (n = 110) | LIS (n = 219) |

Age, years

| Mean (SD) | 28.8 | (10.9) | 29.4 | (10.3) |
|---|---|---|---|---|
| Median | 27.0 | | 28.0 | |
| >18 years | 94 | (85.5%) | 184 | (84.0%) |
| Male Sex, N (%) | 63 | (57.3%) | 114 | (52.1%) |
| US Patients, N (%) | 98 | (89.1%) | 193 | (88.1%) |

FEV₁ percent predicted

| Mean (SD) | 56.3 | (15.9) | 56.6 | (15.7) |
|---|---|---|---|---|
| Median | 57.6 | | 57.3 | |
| <55, N (%) | 52 | (47.3%) | 100 | (45.7%) |

BMI, kg/m2

| | | | | |
|---|---|---|---|---|
| Mean (SD) | 22.1 | (3.79) | 22.6 | (3.95) |

| | | |
|---|---|---|
| Median | 21.7 | 21.8 |

Inhaled antibiotic courses during previous year

| Mean (SD) | 6.0 | (2.77) | 5.9 | (2.65) |
|---|---|---|---|---|
| Median | 6.0 | | 6.0 | |
| <2, N (%) | 5 | (4.5%) | 14 | (6.4%) |
| 3, N (%) | 13 | (11.8%) | 26 | (11.9%) |
| 4, N (%) | 19 | (17.3%) | 25 | (11.4%) |
| 5, N (%) | 7 | (6.4%) | 27 | (12.3%) |
| >6, N (%) | 66 | (60.0%) | 127 | (58.0%) |

Prior-year pulmonary exacerbations

| | | | | |
|---|---|---|---|---|
| 0, N (%) | 21 | (19.1%) | 44 | (20.0%) |
| 1-2, N (%) | 67 | (60.9%) | 101 | (45.9%) |
| >3, N (%) | 22 | (20.0%) | 75 | (34.1%) |

Baseline pathogen isolation, N (%)

| | | | | |
|---|---|---|---|---|
| P aeruginosa | 105 | (95.5%) | 211 | (96.3%) |
| S aureus | 58 | (52.7%) | 110 | (50.2%) |
| Methicillin resistant | 22 | (20.0%) | 56 | (25.6%) |
| S aureus | | | | |
| S maltophilia | 10 | (9.1%) | 17 | (7.8%) |
| A xylosoxidans | 8 | (7.3%) | 7 | (3.2%) |
| B cepacia complex | 3 | (2.7%) | 4 | (1.8%) |

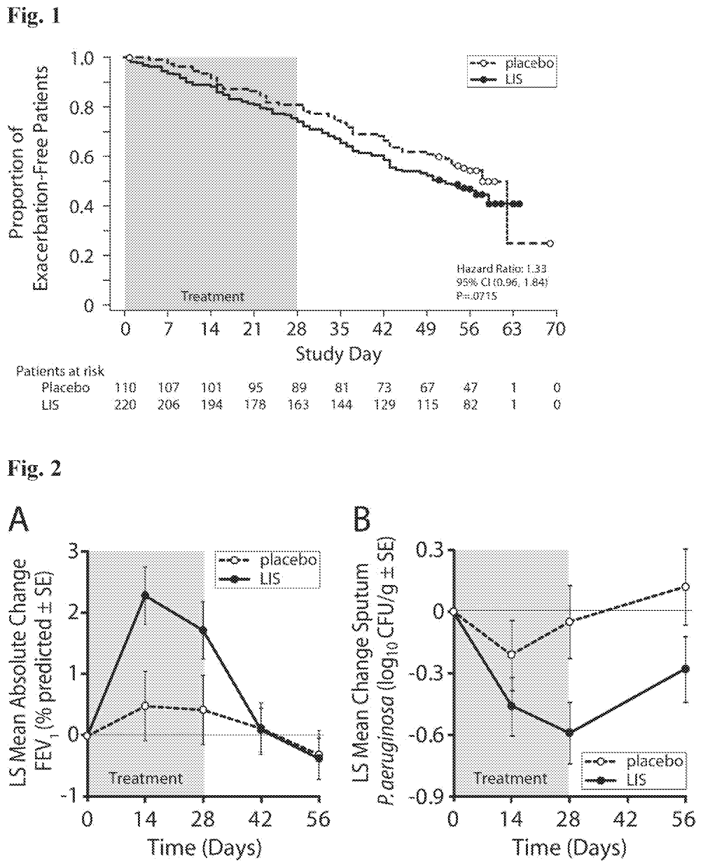

Time to exacerbation. During the study period, 55.5% of patients receiving levofloxacin inhalation solution and 47.3% of patients receiving placebo experienced a protocol-defined pulmonary exacerbation (FIG. 1). There was no statistically significant difference in time to protocol-defined pulmonary exacerbations between treatment arms (HR=1.33, 95%, CI: 0.96-1.84). Most patients who met the protocol definition of an exacerbation did so as a result of concurrent changes in at least 4 of the 12 Fuchs criteria (86.1% of levofloxacin inhalation solution and 84.6% of placebo patients). The remaining patients considered to have had an exacerbation either discontinued from the study early or received an antipseudomonal agent for an event that did not meet the protocol-defined pulmonary exacerbation but was determined to be an exacerbation for the purposes of the primary endpoint by the independent, blinded, exacerbation adjudication committee.

In all, 15.9% of levofloxacin inhalation solution patients and 12.7% of placebo patients met the protocol-defined pulmonary exacerbation endpoint but did not receive antipseudomonal agents within 14 days before or after meeting criteria. Conversely, 15.0% of levofloxacin inhalation solution and 25.5% of placebo patients received an antipseudomonal agent but did not meet the protocol-defined pulmonary exacerbation endpoint prior to or within 14 days after receiving an antipseudomonal agent (based on Blinded Exacerbation Adjudication Committee Summaries).

Pulmonary function. FEV₁ percent predicted values were similar in the treatment groups at Baseline. The LS means for absolute change in FEV₁ percent predicted from Baseline to Day 28 in the ITT population showed an increase in both treatment groups (FIG. 2A). The difference between the treatment groups favored the levofloxacin inhalation solution group, with an LS mean difference of 1.31 (p=0.0137; FIG. 2A). The LS means for relative change in FEV₁ percent predicted from Baseline to Day 28 in the ITT population showed a difference between the treatment groups favoring the levofloxacin inhalation solution group, with an LS mean difference of 2.42 (p=0.011).

Time to antibiotic requirement and hospitalization. The median time to administration of systemic and/or inhaled antipseudomonal antimicrobials for patients who met symptoms requirements at the time of administration of the antipseudomonal antimicrobials was 59 days in the levofloxacin inhalation solution and 58 days in the placebo group in the ITT population. There was no difference in the distribution of time to administration of the antipseudomonal antimicrobials between levofloxacin inhalation solution and placebo (HR=0.85; 95% CI: 0.61, 1.18), and no difference in the time to administration of IV antipseudomonal antimicrobials when symptoms requirements were met between levofloxacin inhalation solution and placebo (HR=0.90; 95% CI: 0.46, 1.78).

Change in Pseudomonas aeruginosa Sputum Density. At Day 14, both treatment groups showed a mean reduction in Pseudomonas aeruginosa sputum density. However, at Day 28, the levofloxacin inhalation solution group showed a mean reduction in Pseudomonas aeruginosa sputum density while the placebo group showed a slight mean increase back to baseline (FIG. 2B). The difference in change from baseline to Day 28 between the treatment groups was an LS mean difference of −0.63 log 10 CFU/g sputum favoring the levofloxacin inhalation solution group (p<0.001; FIG. 2B).

CFQ-R Respiratory Domain. Scores on the Respiratory Domain of the CFQ-R were similar in the treatment groups at Baseline and both treatment groups had a similar mean increase in CFQ-R Respiratory Domain score from Baseline to Day 28. The results were similar at all time points. The between-group difference was not significant in the ITT population.

Discussion. The study did not achieve its primary endpoint of demonstrating superiority of levofloxacin inhalation solution over placebo in the time to a modified pulmonary exacerbation, but did show superiority over placebo in key secondary endpoints including improvement in lung function (FEV₁% predicted) and reduction in bacterial density in the sputum. These latter observations are consistent with other studies of levofloxacin inhalation solution compared to placebo and tobramycin inhalation solution in the treatment of subjects with cystic fibrosis and chronic Pseudomonas aeruginosa infection. The former observation was an unexpected finding given the previous demonstration of a reduction in need for antipseudomonal antibiotics for those treated with levofloxacin inhalation solution compared to those on placebo and the similarity in time to exacerbation in the comparison of levofloxacin inhalation solution to tobramycin inhalation solution.

There are several possibilities that could explain why the primary endpoint of time to a modified pulmonary exacerbation was not met. These include: the treated patients did not receive sufficient concentrations of the antibiotic (analysis of serum levofloxacin concentrations do not support this, data not provided), the drug lacks efficacy (improvement in the other key endpoints does not support this), the study populations are not actually similar, or that the pulmonary exacerbation definition used in the primary endpoint was inappropriate. Further examination of the latter two possibilities was the justification for post hoc analyses.

There is general agreement that a validated pulmonary exacerbation definition does not exist. Other studies have used clinical criteria (i.e. signs and symptoms) or the use of antibiotics as clinical endpoints. For this study, a modified pulmonary exacerbation endpoint was used, although this clinical endpoint has not been validated. The original definition of pulmonary exacerbation required a clinician's decision to treat a patient (e.g., with IV antibiotics) and the presence of at least 4 of the 12 Fuchs criteria. If study patients were not treated with IV antibiotics, or if they were treated but less than 4 of the 12 Fuchs criteria were present, the definition of pulmonary exacerbation was not met in this study. Importantly, this pulmonary exacerbation definition is not used clinically in the management of patients with cystic fibrosis. In contrast, the modified pulmonary exacerbation endpoint drops the requirement for antibiotic treatment and limits the definition to meeting at least 4 of the 12 Fuchs criteria. A previous post hoc analysis of the data from this study as well as another study comparing levofloxacin inhalation solution to tobramycin inhalation solution demonstrated that the modified pulmonary exacerbation endpoint was a poor predictor for whether a clinician would ultimately treat with antibiotics. Of 310 participates meeting the modified pulmonary exacerbation endpoint in these two studies, only 172 (55.5%) were also treated with antibiotics within 14 days before or after the event. Given that this current application of the modified pulmonary exacerbation endpoint is outside of the context in which it was originally developed and has never been validated, it is reasonable to conclude that a therapy that significantly reduces the risk of a pulmonary exacerbation diagnosis would not necessarily be identified by the modified pulmonary exacerbation endpoint.

Rather than using the time to exacerbation by the modified pulmonary exacerbation endpoint, it is prudent to consider time to antibiotic treatment, a subjective but direct measure of clinician exacerbation diagnosis and which is clinically relevant because it results in a clinical action being taken. It is noted that in another study, participants randomized to levofloxacin inhalation solution had a significantly lower risk of treatment with antibiotics for an exacerbation than did those participants treated with tobramycin inhalation solution. Although the comparison of levofloxacin inhalation solution to placebo in this study improved when time to antibiotic treatment for exacerbation was considered in place of the modified pulmonary exacerbation definition, the treatment effect remained statistically non-significant. Among patients receiving levofloxacin inhalation solution, 109 (49.5%) were treated with antibiotics for exacerbation during the study, compared with 62 (56.4%) patients receiving placebo (P=0.293).

Recent observations in analyses of pulmonary exacerbations have demonstrated that one of the strongest predictors of hazard for treatment with IV antibiotics for pulmonary exacerbations among cystic fibrosis patients is the number of such treatments received in the prior year. Patients who experienced 3 or more treatments for pulmonary exacerbation in the prior year had a greater than 25 fold increased hazard of future treatment compared to patients who were not treated in the prior year, while patients treated once or twice in the prior year had a greater than 4 fold increased hazard. VanDeVanter et al, Journal of Cystic Fibrosis, 14:763-769 (2015); VanDevanter et al, Journal of Cystic Fibrosis, 15(3):372-379 (2016). Although the patient characteristics of both treatment groups for this study were generally similar, one clear baseline difference among groups was the percentage of patients with 3 or more pulmonary exacerbations in the prior year (20% placebo vs. 34.1% levofloxacin inhalation solution, P=0.011; Table 1). This disproportionate allocation of patients at highest risk for pulmonary exacerbation suggests that the levofloxacin inhalation solution group was at a greater overall hazard for pulmonary exacerbation at baseline compared to the placebo group. When the proportion of patients either meeting the modified pulmonary exacerbation endpoint or treated with antibiotics for exacerbation are compared between treatment groups but within subgroups with similar exacerbation risk, as predicted by prior-year exacerbation incidents, an interesting picture emerges, as shown in Table 2. In addition to quantifying the extent of disproportionate allocation between treatment groups, this post hoc analysis shows that a levofloxacin inhalation solution treatment effect (as measured by hazard of antibiotic treatment for pulmonary symptoms) was more likely to be observed among patients at higher risk for exacerbation at study entry. For instance, the hazard ratio for antibiotic treatment (levofloxacin inhalation solution/placebo) among patients with ≥3 prior-year exacerbations was 0.56 (95% confidence interval 0.30, 1.05; log rank P=0.028; Table 3), compared with 0.79 (0.51, 1.23; P=0.285) for those with 1 or 2 prior-year exacerbations and 1.34 (0.55, 3.30; P=0.586) for those with no prior-year exacerbations (Table 2). A comparable relationship between prior-year levofloxacin inhalation solution exacerbation group and apparent levofloxacin inhalation solution treatment effect was not observed for the modified pulmonary exacerbation endpoint (Table 2).

TABLE 2
Patients Meeting the Modified Pulmonary Exacerbation
(PE) Endpoint or Treated with Antibiotics for Pulmonary
Symptoms by Prior-Year Exacerbation History.

| | | | | | Hazard | Log |
|---|---|---|---|---|---|---|
| | Placebo | | LIS | | Ratio | Rank |
| All Patients, N | 110 | | 220 | | | |
| Met Modified | 44 | (40.0%) | 108 | (49.1%) | 1.35 [0.97, 1.89] | .0871 |
| PE Endpoint, | | | | | | |
| N(%) | | | | | | |
| Treated with | 62 | (56.4%) | 109 | (49.5%) | 0.84 [0.61, 1.16] | .2708 |
| Antibiotics, | | | | | | |
| N(%) | | | | | | |
| No Prior-Year | 21 | (19.1%) | 44 | (20.0%) | | |
| Exacerbations, | | | | | | |
| N(%) | | | | | | |
| Met Modified | 4 | (19.0%) | 18 | (40.9%) | 2.77 [1.17, 6.55] | .0525 |
| PE Endpoint, | | | | | | |
| N(%) | | | | | | |
| Treated with | 6 | (28.6%) | 15 | (34.1%) | 1.34 [0.55, 3.30] | .5364 |
| Antibiotics, | | | | | | |
| N(%) | | | | | | |
| 1-2 Prior-Year | 67 | (60.9%) | 101 | (45.9%) | | |
| Exacerbations, | | | | | | |
| N(%) | | | | | | |
| Met Modified | 30 | (44.8%) | 45 | (44.6%) | 0.97 [0.61, 1.55] | .9066 |
| PE Endpoint, | | | | | | |
| N(%) | | | | | | |
| Treated with | 37 | (55.2%) | 48 | (47.5%) | 0.79 [0.51, 1.23] | .2853 |
| Antibiotics, | | | | | | |
| N(%) | | | | | | |
| ≥3 Prior-Year | 22 | (20.0%) | 75 | (34.1%) | | |
| Exacerbations, | | | | | | |
| N(%) | | | | | | |
| Met Modified | 10 | (45.5%) | 45 | (60.0%) | 1.42 [0.77, 2.64] | .3054 |
| PE Endpoint, | | | | | | |
| N(%) | | | | | | |
| Treated with | 19 | (86.4%) | 46 | (61.3%) | 0.56 [0.30, 1.05] | .0281 |
| Antibiotics, | | | | | | |
| N(%) | | | | | | |

The Hazard Ratio is based on levofloxacin inhalation solution (LIS)/Placebo, Kaplan Meier survival method.

In this study, LIS demonstrated clinical efficacy by a reduction in bacterial density and an increase in lung function. In those patients with a prior history of frequent exacerbations, it increased the time to antibiotic treatment for future pulmonary exacerbations. Given its proven safety and tolerability record, levofloxacin inhalation solution demonstrates effective therapy for some patients with cystic fibrosis and chronic Pseudomonas aeruginosa infection of the airways, particularly those patients with 3 or more pulmonary exacerbations in the prior year.

### Example 3

Levofloxacin inhalation solution was approved by the EMA after a three on/off cycle 168-day study demonstrated clinical non-inferiority to tobramycin inhalation solution. A concern with extended levofloxacin inhalation solution use is a potential for reduction in levofloxacin-susceptible Pseudomonas aeruginosa isolate prevalence. We describe airway bacterial culture and Pseudomonas aeruginosa isolate antimicrobial susceptibilities at baseline and study end for levofloxacin inhalation solution (N=189) and tobramycin inhalation solution (N=93) subjects. In this example “levofloxacin inhalation solution” refers to an aerosol of a solution containing about 100 mg/ml of levofloxacin and about 200 mM magnesium chloride.

Sputum or throat swabs were cultured for Pseudomonas aeruginosa and other cystic fibrosis airway bacterial opportunists. Multiple Pseudomonas aeruginosa morphotypes were isolated when present and susceptibility was tested using microdilution with EUCAST breakpoints.

No statistically significant change in species prevalence from baseline or changes in prevalence of Pseudomonas aeruginosa isolates susceptible to levofloxacin or tobramycin were observed: 34% [95% CI 28-39%] of levofloxacin inhalation solution isolates and 43[35-51]% of tobramycin inhalation solution isolates were levofloxacin-susceptible at baseline; 64[59-70]% of levofloxacin inhalation solution isolates and 66[58-73]% of tobramycin inhalation solution isolates were tobramycin-susceptible at baseline. At study end, 24[20-29]% of levofloxacin inhalation solution isolates and 31[24-39]% of tobramycin inhalation solution isolates were levofloxacin-susceptible; 63[57-68]% of levofloxacin inhalation solution and 62[53-69]% of tobramycin inhalation solution isolates were tobramycin-susceptible. Prevalence of isolates not susceptible to any agent from 23 antipseudomonal classes did not differ from baseline (levofloxacin inhalation solution 40[35-56]%, tobramycin inhalation solution 43[35-51]%) to study end (levofloxacin inhalation solution 42[36-47]%, tobramycin inhalation solution 46[38-54]%).

While the clinical utility of susceptibility testing in cystic fibrosis is uncertain, patients treated with levofloxacin inhalation solution vs. tobramycin inhalation solution had no meaningful differences in microbial prevalence or Pseudomonas aeruginosa isolate antimicrobial susceptibilities at baseline or at treatment end.

While embodiments have been shown and described herein, it will be obvious to those skilled in the art that such embodiments are provided by way of example only. Numerous variations, changes, and substitutions will occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for treating a Pseudomonas aeruginosa pulmonary infection in a high-risk cystic fibrosis patient in need thereof comprising administering a therapeutically effective amount of an aerosol of a solution comprising levofloxacin to the high-risk cystic fibrosis patient.

2. A method for reducing the incidents of a pulmonary exacerbation in a high-risk cystic fibrosis patient in need thereof comprising administering a therapeutically effective amount of an aerosol of a solution comprising levofloxacin to the high-risk cystic fibrosis patient.

3. A method for extending the time between the onset of pulmonary exacerbations in a high-risk cystic fibrosis patient in need thereof comprising administering a therapeutically effective amount of an aerosol of a solution comprising levofloxacin to the high-risk cystic fibrosis patient.

4. A method for treating a pulmonary bacterial infection in a high-risk cystic fibrosis patient in need thereof comprising administering a therapeutically effective amount of an aerosol of a solution comprising levofloxacin to the high-risk cystic fibrosis patient.

5. A method of treating a pulmonary exacerbation in a high-risk cystic fibrosis patient in need thereof comprising administering a therapeutically effective amount of an aerosol of a solution comprising levofloxacin to the high-risk cystic fibrosis patient.

6. A method of treating cystic fibrosis in a high-risk cystic fibrosis patient in need thereof comprising administering a therapeutically effective amount of an aerosol of a solution comprising levofloxacin to the high-risk cystic fibrosis patient.

7. A method of reducing a Pseudomonas aeruginosa sputum density in a high-risk cystic fibrosis patient in need thereof comprising administering a therapeutically effective amount of an aerosol of a solution comprising levofloxacin to the high-risk cystic fibrosis patient.

8. The method of any one of claims 1-7, wherein the high-risk patient had at least 3 occurrences of a pulmonary exacerbation in the prior year.

9. The method of any one of claims 1-7, wherein the high-risk patient had at least 4 occurrences of a pulmonary exacerbation in the prior year.

10. The method of any one of claims 1-7, wherein the high-risk patient had at least 5 occurrences of a pulmonary exacerbation in the prior year.

11. The method of any one of claims 2, 3, 5, and 8-10, wherein the pulmonary exacerbation comprises at least four of the following: (i) a change in sputum; (ii) a new or increased hemoptysis; (iii) an increased cough; (iv) increased dyspnea; (v) malaise, fatigue, or lethargy; (vi) a temperature above 38° C.; (vii) anorexia or weight loss; (viii) sinus pain or tenderness; (ix) a change in sinus discharge; (x) a change in physical examination of the chest; (xi) a decrease in pulmonary function by 10% or more from a previously recorded value; and (xii) a radiographic change indicative of pulmonary infection.

12. The method of any one of claims 2, 3, 5, and 8-10, wherein the pulmonary exacerbation is a pulmonary bacterial infection.

13. The method of any one of claims 2, 3, 5, and 8-10, wherein the pulmonary exacerbation is a Pseudomonas aeruginosa pulmonary infection.

14. The method of any one of claims 2, 3, 5, and 8-10, wherein the pulmonary exacerbation is a Pseudomonas pulmonary infection.

15. The method of any one of claims 2, 3, 5, and 8-10, wherein the pulmonary exacerbation is a Staphylococcus aureus pulmonary infection.

16. The method of any one of claims 2, 3, 5, and 8-10, wherein the pulmonary exacerbation is a Staphylococcus pulmonary infection.

17. The method of any one of claims 2, 3, 5, and 8-10, wherein the pulmonary exacerbation is a decrease of 10% or more in the FEV₁ of the predicted value using Hankinson/NHANES III reference equation.

18. The method of any one of claims 1-17, wherein the high-risk patient has a forced expiratory volume in 1 second (FEV₁) between 25 and 85 percent of their predicted values using Hankinson/NHANES III reference equation.

19. The method of any one of claims 1-17, wherein the high-risk patient had a chronic airway infection with Pseudomonas aeruginosa and had received at least three 28-day courses of an inhaled tobramycin formulation in the prior year.

20. The method of any one of claims 1-17, wherein the high-risk patient had a chronic airway infection with Pseudomonas aeruginosa and had received at least three 28-day courses of an inhaled aminoglycoside formulation in the prior year, an inhaled monobactam formulation in the prior year, an inhaled polymyxin formulation in the prior year, or a combination of two or more thereof.

21. The method of any one of claims 1-20, wherein the solution comprises levofloxacin or ofloxacin and a divalent or trivalent cation.

22. The method of claim 21, wherein the trivalent cation is aluminum or iron.

23. The method of any one of claims 1-20, wherein the solution comprises levofloxacin and a divalent cation.

24. The method of claim 23, wherein the divalent cation is magnesium, calcium, zinc, copper, or iron.

25. The method of claim 23, wherein the divalent cation is magnesium.

26. The method of any one of claims 1-20, wherein the solution comprises about 75 mg/ml to about 150 mg/ml of levofloxacin and about 150 mM to about 250 mM of a divalent cation.

27. The method of claim 26, wherein the divalent cation is magnesium, calcium, zinc, copper, or iron.

28. The method of claim 26, wherein the divalent cation is magnesium.

29. The method of any one of claims 1-20, wherein the solution comprises about 90 mg/ml to about 110 mg/ml of levofloxacin and about 190 mM to about 210 mM of a divalent cation.

30. The method of claim 29, wherein the divalent cation is magnesium, calcium, zinc, copper, or iron.

31. The method of claim 29, wherein the divalent cation is magnesium.

32. The method of any one of claims 1-20, wherein the solution comprises about 80 mg/ml to about 120 mg/ml of levofloxacin and about 180 mM to about 220 mM of a divalent cation.

33. The method of claim 32, wherein the divalent cation is magnesium, calcium, zinc, copper, or iron.

34. The method of claim 32, wherein the divalent cation is magnesium.

35. The method of any one of claims 1-20, wherein solution comprises about 100 mg/ml of levofloxacin and about 200 mM magnesium chloride.

36. A method for treating a Pseudomonas aeruginosa pulmonary infection in a high-risk cystic fibrosis patient in need thereof comprising administering a therapeutically effective amount of an aerosol of a solution comprising ofloxacin to the high-risk cystic fibrosis patient.

37. A method for reducing the incidents of a pulmonary exacerbation in a high-risk cystic fibrosis patient in need thereof comprising administering a therapeutically effective amount of an aerosol of a solution comprising ofloxacin to the high-risk cystic fibrosis patient.

38. A method for extending the time between the onset of pulmonary exacerbations in a high-risk cystic fibrosis patient in need thereof comprising administering a therapeutically effective amount of an aerosol of a solution comprising ofloxacin to the high-risk cystic fibrosis patient.

39. A method for treating a pulmonary bacterial infection in a high-risk cystic fibrosis patient in need thereof comprising administering a therapeutically effective amount of an aerosol of a solution comprising ofloxacin to the high-risk cystic fibrosis patient.

40. A method of treating a pulmonary exacerbation in a high-risk cystic fibrosis patient in need thereof comprising administering a therapeutically effective amount of an aerosol of a solution comprising ofloxacin to the high-risk cystic fibrosis patient.

41. A method of treating cystic fibrosis in a high-risk cystic fibrosis patient in need thereof comprising administering a therapeutically effective amount of an aerosol of a solution comprising ofloxacin to the high-risk cystic fibrosis patient.

42. A method of reducing a Pseudomonas aeruginosa sputum density in a high-risk cystic fibrosis patient in need thereof comprising administering a therapeutically effective amount of an aerosol of a solution comprising ofloxacin to the high-risk cystic fibrosis patient.

43. The method of any one of claims 36-42, wherein the high-risk patient had at least 3 occurrences of a pulmonary exacerbation in the prior year.

44. A method for treating a Pseudomonas aeruginosa pulmonary infection in a high-risk cystic fibrosis patient in need thereof comprising administering a therapeutically effective amount of an aerosol of a solution comprising a fluoroquinolone to the high-risk cystic fibrosis patient.

45. A method for reducing the incidents of a pulmonary exacerbation in a high-risk cystic fibrosis patient in need thereof comprising administering a therapeutically effective amount of an aerosol of a solution comprising a fluoroquinolone to the high-risk cystic fibrosis patient.

46. A method for extending the time between the onset of pulmonary exacerbations in a high-risk cystic fibrosis patient in need thereof comprising administering a therapeutically effective amount of an aerosol of a solution comprising a fluoroquinolone to the high-risk cystic fibrosis patient.

47. A method of reducing a Pseudomonas aeruginosa sputum density in a high-risk cystic fibrosis patient in need thereof comprising administering a therapeutically effective amount of an aerosol of a solution comprising a fluoroquinolone to the high-risk cystic fibrosis patient.

48. A method for treating a pulmonary bacterial infection in a high-risk cystic fibrosis patient in need thereof comprising administering a therapeutically effective amount of an aerosol of a solution comprising a fluoroquinolone to the high-risk cystic fibrosis patient.

49. A method of treating a pulmonary exacerbation in a high-risk cystic fibrosis patient in need thereof comprising administering a therapeutically effective amount of an aerosol of a solution comprising a fluoroquinolone to the high-risk cystic fibrosis patient.

50. A method of treating cystic fibrosis in a high-risk cystic fibrosis patient in need thereof comprising administering a therapeutically effective amount of an aerosol of a solution comprising a fluoroquinolone to the high-risk cystic fibrosis patient.

51. The method of any one of claims 44-50, wherein the fluoroquinolone is ciprofloxacin, enoxacin, gatifloxacin, gemifloxacin, lomefloxacin, moxifloxacin, norfloxacin, pefloxacin, sparfloxacin, garenoxacin, sitagloxacin, or DX-619.

52. The method of any one of claims 44-50, wherein the high-risk patient had at least 3 occurrences of a pulmonary exacerbation in the prior year.

53. A method for treating a Pseudomonas aeruginosa pulmonary infection in a high-risk cystic fibrosis patient in need thereof comprising administering by inhalation a therapeutically effective amount of a pharmaceutical composition comprising an antibiotic to the high-risk cystic fibrosis patient.

54. A method for reducing the incidents of a pulmonary exacerbation in a high-risk cystic fibrosis patient in need thereof comprising administering by inhalation a therapeutically effective amount of a pharmaceutical composition comprising an antibiotic to the high-risk cystic fibrosis patient.

55. A method for extending the time between the onset of pulmonary exacerbations in a high-risk cystic fibrosis patient in need thereof comprising administering by inhalation a therapeutically effective amount of a pharmaceutical composition comprising an antibiotic to the high-risk cystic fibrosis patient.

56. A method for treating a pulmonary bacterial infection in a high-risk cystic fibrosis patient in need thereof comprising administering by inhalation a therapeutically effective amount of a pharmaceutical composition comprising an antibiotic to the high-risk cystic fibrosis patient.

57. A method of treating a pulmonary exacerbation in a high-risk cystic fibrosis patient in need thereof comprising administering by inhalation a therapeutically effective amount of a pharmaceutical composition comprising an antibiotic to the high-risk cystic fibrosis patient.

58. A method of treating cystic fibrosis in a high-risk cystic fibrosis patient in need thereof comprising administering by inhalation a therapeutically effective amount of a pharmaceutical composition comprising an antibiotic to the high-risk cystic fibrosis patient.

59. The method of any one of claims 53-58, wherein the high-risk patient had at least 3 occurrences of a pulmonary exacerbation in the prior year.

60. The method of any one of claims 53-59, wherein the antibiotic is tobramycin.

61. The method of any one of claims 53-59, wherein the antibiotic is aztreonam.

62. The method of claim 61, wherein the aztreonam is aztreonam lysine.
